(19) United States
(12) Patent Application Publication
(10) Pub. No.: US 2008/0156328 A1
(43) Pub. Date: Jul. 3, 2008

(54) SOLENOID AIR/OXYGEN SYSTEM FOR USE WITH AN ADAPTIVE OXYGEN CONTROLLER AND THERAPEUTIC METHODS OF USE

(76) Inventor: John TAUBE, Oreland, PA (US)

Correspondence Address: The Law Office of Paul Roath, P.O. Box 427, Norristown, PA, 19404, US

(21) Appl. No.: 11/938,289

(22) Filed: Nov. 11, 2007

Related U.S. Application Data
(60) Provisional application No. 60/858,483, filed on Nov. 13, 2006.

(51) Int. Cl. A61M 16/12

(52) U.S. Cl. 128/204.22; 128/204.21

(57) ABSTRACT

A pair of solenoid air/oxygen mixing systems used by an adaptive controller for delivering fractional inspired oxygen to a patient is described. The solenoid control system comprises either a bi-modal solenoid or a proportional solenoid air/oxygen mixing system to derive a fraction of inspired oxygen delivered to a patient. In the bi modal solenoid air/oxygen mixing system, a derived fraction of insipid oxygen is delivered to a patient. The bi-modal mixer uses a three-way valve solenoid. The solenoid has two input gas ports and one output gas port. Toggling between the two input port gases generates an output gas oxygen concentration. In contrast with the bi-modal solenoid, variation or proportionality between the two input port gases generates an output gas oxygen concentration. Both solenoid systems use a mini-computer and digital controller with software to control the fraction of inspired oxygen delivered to a patient. Finally, several therapeutic applications are described.

### CROSS-REFERENCE TO RELATED APPLICATIONS

This application claims priority to U.S. Provisional Application No. 60/858,483 filed on Nov. 13, 2006, the entire contents of which are incorporated herein by reference.

### DESCRIPTION

### Background of the Invention

This invention relates to a solenoid mixing system that integrates an adaptive oxygen control system utilizing supplemental oxygen (SpO₂) feedback for calculating the fraction of inspired supplemental oxygen delivered to a patient. The adaptive oxygen system is well known. U.S. Pat. No. 4,889,116 issued to John Taube on Dec. 26, 1989 shows a method and apparatus for the adaptive control of oxygen by using SPO₂ feedback. This invention also relates generally to continuous positive airway pressure ventilation systems such as respirators and a useful non-invasive adaptive controller of blood system oxygen. The system has particular application in the adaptive control of fractional inspired oxygen (FiO₂) and is intended to make more automatic the control of oxygen to the patient regardless of the patent's age.

This system further utilizes a pulse oximeter to optically determine hemoglobin saturation of the patient's blood and use this information to regulate oxygen delivered to the patient's breathing mask or hood. The control mechanism is derived from the known relationship between the minimum required FiO₂ delivered to the patient and predetermined lung function dynamics in order to maintain a desirable arterial blood hemoglobin saturation level (HSAT).

The use of a pulse oximeter permits non-invasive determination of a patient's arterial blood hemoglobin saturation and pulse rate. From the measured hemoglobin saturation and pulse rate a non-invasive determination of pulse rate and blood pressure parameters can be used to determine patient movement and apnea to suspend and correct, respectively, the operation of the system without requiring operator intervention.

The prior art is, however, is devoid of solenoid mixing systems that utilizes either a bi-modal mixing system or variable modal mixing system that precisely generates an output gas concentration by using two input gases of 21% and 100% oxygen. A bi-modal solenoid mixer is vital to the mixing system in that it quickly and precisely controls the percentage of the output oxygen concentration by changing the toggle frequency between the two input gases. The output gas mixture being of a pulsatile nature requires a mixing chamber, which is vital to ensure complete gas mixing. The proportional solenoid mixing system, using precisely tuned input gas pressures, to precisely generate an output gas concentration by using two input gases of 21% and 100% oxygen. A proportional solenoid mixer is important to the mixing system in that it quickly and precisely controls the percentage of the output oxygen concentration by changing the variation or proportionality between the two input gases and eliminates the need of a mixing chamber that is required for the bi-modal mixing system to control the pulse that is a natural bi-product of the bi-modal mixing system.

The present invention also provides therapeutic uses for the solenoid mixing system. In particular, the therapeutic applications include sleep apnea therapy, long-term supplemental oxygen therapy as well as the weaning of patients from long-term supplemental oxygen therapy through the gradual reduction of supplemental oxygen over the normal 21% O₂ found in air. Another therapeutic application of the present invention provides for a helium-oxygen mix.

### DESCRIPTION OF THE PRIOR ART

The idea of continuous oxygen flow adjustment to maintain patient saturation has existed for over 50 years. U.S. Pat. No. 2,414,747 by Kirschbaum (1947) discloses a method and apparatus for controlling oxygen content of the blood of living animal. The method used an ear oximeter, which produced a signal to control the fraction of inspired oxygen (Fl0₂). U.S. Pat. No. 4,889,116 by Taube in 1986 describes an adaptive controller, which utilizes a pulse oximeter to measure blood oxygen saturation (SpO₂). This measurement would be used to calculate the necessary FiO₂ to maintain a preset saturation level.

U.S. Pat. No. 5,365,922 by Raemer describes a closed loop non-invasive oxygen saturation control system which uses an adaptive controller for delivering a fractional amount of oxygen to a patient. Features of the control algorithm include a method for recognizing when pulse oximeter values deviate significantly from what should be expected. At this point the controller causes a gradual increase in the fractional amount of oxygen delivered to the patient. The feedback control means is also disconnected periodically and the response of the patient to random changes in the amount of oxygen delivered is used to tune the controller response parameters.

U.S. Pat. No. 5,682,877 describes a system and method for automatically selecting an appropriate oxygen dose to maintain a desired blood oxygen saturation level is disclosed. The system and method are particularly suited for use with ambulatory patients having chronic obstructive lung disease or other patients requiring oxygenation or ventilation. In one embodiment, the method includes delivering a first oxygen dose to the patient while repeatedly sequencing through available sequential oxygen doses at predetermined time intervals until the current blood oxygen saturation level of the patent attains the desired blood oxygen saturation levels. The method then continues with delivering the selected oxygen dose to the patient so as to maintain the desired blood oxygen saturation level.

U.S. Pat. No. 6,192,883 B1 describes an oxygen control system for supplying a predetermined rate of flow from an oxygen source to a person in need of supplemental oxygen comprising in input manifold, an output manifold and a plurality of gas conduits interconnecting the input manifold to the output manifold. The oxygen source is arranged in flow communication with the input manifold, and a needle valve is positioned in flow control relation to each of the conduits so as to control the flow of oxygen from the input manifold to the output manifold. A plurality of solenoid valves, each having a first fully closed state corresponding to a preselected level of physical activity of the person and a second, fully open state corresponding to another preselected level of physical activity of the person, are positioned in flow control relation to all but one of the conduits. Sensors for monitoring the level of physical activity of the person are provided, along with a control system that is responsive to the monitored level of physical activity, for switching the solenoids between the first state and the second state. A method for supplying supplemental oxygen to a person according to the level of physical activity undertaken by that person is also provided.

World Patent application No. WO _{02/056931} A2 by Tyomkin, et al. describes a method for controlling flow of gas to a patient by measuring of a preselected dissolved substance in the blood stream of a patient. The amount of gas is regulated to maintain the preselected dissolved substance above a desired value.

U.S. Pat. No. 7,206,621 issued to T. Aoyagi, et al, describes a pulse oxymeter which can measure an oxygen saturation of arterial blood continuously and non-invasively by utilization of variations in the volume of arterial blood by pulsation. Numerous improvements have been made since that time wherein better matching of oxygen delivery to the needs of the patient have been made such as shown in U.S. Pat. No. 3,734,091 to Ronald H. Taplin issued on May 22, 1973. Taplin discloses an optical oximeter and a temporary oxygen-deficient mixture (anoxic) to control blood oxygen saturation. Thus, to prevent super saturation, or more than 100% oxygen saturation, Taplin discloses limiting the oxygen by proving the anoxic mixture each time the saturation of the blood reaches a predetermined percentage level.

An invasive patient data controlled respiration system is shown in U.S. Pat. No. 4,326,513 of Volker Schultz, et al, issued on Apr. 27, 1982 which shows a patient data controlled respiration system utilizing sensed concentration of oxygen in the patient's blood to control a respirator supplying breathing air having the selected concentration of oxygen to the patient. In such a system, a sensor is connected to the patient for sensing arterial partial pressure of the patient's blood (PaO₂). The system further includes a minimizing comparator which has preset threshold levels and determines whether the FiO₂ value is above or below those threshold values. When a transient FiO₂ value rises above or drops below the threshold value, it causes the control device to cancel the adjustment to the inspired oxygen and causes the previous amount of oxygen to be supplied to the patient. In this way, there can be only small changes in the original FiO₂. Disruptions of the respiratory system during sleep may include the conditions of sleep apnea or sleep hypopnea. Sleep apnea is a serious breathing disorder caused by airway obstruction, denoted obstructive sleep apnea, or derangement in central nervous system control of respiration, denoted central sleep apnea. Regardless of the type of apnea, people with sleep apnea stop breathing repeatedly during their sleep, sometimes hundreds of times a night and often for a minute or longer. Whereas sleep apnea refers to cessation of breathing, hypopnea is associated with periods of abnormally slow or shallow breathing. With each apnea or hypopnea event, the person generally briefly arouses to resume normal breathing. As a result, people with sleep apnea or hypopnea may experience sleep fragmented by frequent arousals.

Reversible obstructive pulmonary disease includes asthma and reversible aspects of chronic obstructive pulmonary disease (COPD). Asthma is a disease in which (i) bronchoconstriction, (ii) excessive mucus production, and (iii) inflammation and swelling of airways occur, causing widespread but variable. Asthma is a chronic disorder, primarily characterized by persistent airway inflammation. However, asthma is further characterized by acute episodes of additional airway narrowing via contraction of hyper-responsive airway smooth muscle.

The reversible aspects of COPD generally describe excessive mucus production in the bronchial tree. Usually, there is a general increase in bulk (hypertrophy) of the large bronchi and chronic inflammatory changes in the small airways. Excessive amounts of mucus are found in the airways and semisolid plugs of mucus may occlude some small bronchi. Also, the small airways are narrowed and show inflammatory changes. The reversible aspects of COPD include partial airway occlusion by excess secretions, and airway narrowing secondary to smooth muscle contraction, bronchial wall edema and inflation of the airways

Pulmonary diseases, such as chronic obstructive pulmonary disease, (COPD), reduce the ability of one or both lungs to fully expel air during the exhalation phase of the breathing cycle. The term “Chronic Obstructive Pulmonary Disease” (COPD) refers to a group of diseases that share a major symptom, dyspnea. Such diseases are accompanied by chronic or recurrent obstruction to air flow within the lung. Because of the increase in environmental pollutants, cigarette smoking, and other noxious exposures, the incidence of COPD has increased dramatically in the last few decades and now ranks as a major cause of activity-restricting or bed-confining disability in the United States. COPD can include such disorders as chronic bronchitis, bronchiectasis, asthma, and emphysema. While each has distinct anatomic and clinical considerations, many patients may have overlapping characteristics of damage at both the acinar (as seen in emphysema) and the bronchial (as seen in bronchitis) levels.

Helium-oxygen gas mixture (heliox) has been found to be an effective treatment regiment for upper airway obstruction. Additionally, heliox is used to treat a diving condition called “the bends” which occurs when a diver in adequately decompresses from a deep dive.

Research has found a number of disease conditions in which heliox therapy is very effective. Exemplary publications include: T. S. Lu, et al.; Helium/Oxygen in the treatment of upper airway obstruction; Anesthesiology 1976; 45: 678-680; S. T. Shiue, et al.; The use of helium-oxygen mixture in the support of patients with status asthmaticus and respiratory acidosis; J. Asthma 1989; 26: 177-180; J. E. Kass, et al.; Heliox therapy in acute severe asthma; Chest 1995; 107: 757-760; M. R. Wolfson, et al.; Mechanics and energetics of breathing helium in infants with bronchopulmonary dysplasia; J. Pediatr. 1984; 104: 752-757; R. A. Sauder, et al.; Helium-oxygen and conventional mechanical ventilation in the treatment of large airway obstruction and respiratory failure in an infant; South. Med. J.; 1991; 84: 646-648; C. Elleau, et al.; Helium-oxygen mixture in respiratory distress syndrome: a double blind study; J. Pediatr, 1993; 122: 132-136; D. M. Swidwa et al.; Saidel G M; Helium-oxygen breathing in severe chronic obstructive pulmonary disease; Chest 1985; 87: 790-795; C. A. Manthous, et al.; Heliox improves pulsus paradoxus and peak expiratory flow in nonintubated patients with severe asthma; Am. J. Respir. Crit. Care Med; 1995; 151: 310-314; and F. Martin; Utilisation de melanges Helium/Oxygene au cours de letat de mal asthmatique (Use of Helium/Oxygen mixtures during the asthma illness); Rev. Pneumol. Clin. 1987; 43: 186-189. In addition, two publications relate to the use of oxygen/helium mixtures in acute asthma patients, namely: Evaluation of Heliox in children hospitalized with acute severe asthma; Chest 1996; 109: 1256-61, and Kudukis, et al.; Inhaled Helium-oxygen revisited; Effect of inhaled Helium-oxygen during the treatment of status asthmaticus in children; J. Pediatr. 1997; 130: 217-24. The use of a mixture with 80% of helium and 20% of oxygen shows a decrease in the paradoxical pulse rate and an increase in the peak respiratory rate in these patients.

In addition, the document EP-A-741588 describes the use of a gas containing helium and/or neon as medicinal aerosol vector for the treatment of asthma. According to this document, the proportion of helium in the gas is greater than or equal to 70%. It should be noted that similar results had already been obtained and reported by the document M. Svartengren et al.; Human Lung Deposition of Particles Suspended in Air or in Helium/Oxygen Mixture; Exp. Lung. Research, 15: 575-585, 1989; as well as by the publication A. Malanga, et al.; Heliox Improves Rate of Response to aerosol bronchodilator; Am. Review of Resp. Dis.; International Conference Supplement, Vol. 147, No. 4, April 1993, A65.

The prior art is however, devoid of a non-invasive relatively inexpensive system for the control of oxygen delivery which will detect patient movement to suspend adaptive control of the oxygen supplied to the patient and will provide a corrective amount of oxygen to the patient when apnea is detected. Both of these features are vital when controlling oxygen supply to a patent because hypoxia and apnea both can cause irreparable harm to a patent.

### OBJECTS OF THE INVENTION

It is therefore an object of the invention to provide a new and useful solenoid mixing system for use with an adaptive control of fractional inspired oxygen. Another object of the invention is to provide solenoid mixing system that uses two input gases of 21% and 100% oxygen to produce an output gas that varies between 21% and 100% oxygen concentration.

Yet another object of the invention is to provide a bi-modal solenoid mixing system. The bi-modal solenoid mixing system employs a computer to toggle the bi-modal solenoid between the 2 input gases to determine an output gas concentration. The computer uses a SPO₂ feedback to determine the precise oxygen supplemental concentration delivered to a patient.

Another object of the invention is to provide a mixing chamber located between the bi-modal solenoid and the patent ensures complete mixing of an output gas. The mixing chamber eliminates a pulsatile nature of the output gas mixture from the bi-modal solenoid.

Yet another object of the invention is to provide a variable solenoid mixing system. The variable solenoid mixing system utilizes a computer that uses a SpO₂ feedback to determine the precise oxygen supplemental concentration delivered to a patient. The computer varies the variable solenoid between the two input gases determines an output gas concentration.

In yet another object of the invention a variable solenoid mixing system that uses two precisely tuned input pressures to produce an output gas is provided. The input gases comprise 21% and 100% oxygen and the output gas varies between 21% and 100% oxygen concentration.

Another object of the invention is to provide a solenoid mixing system utilizing an adaptive controller for delivering fractional inspired oxygen to a patient. The adaptive controller comprising a pulse oximeter adapted to be connected by an optical sensor to a patient for measuring the patient's blood hemoglobin saturation and pulse rate. The pulse oximeter generates signals representative of said blood hemoglobin saturation and the pulse rate, calculating a means responsive to the signals from the oximeter for determining the fractional inspired oxygen level to be delivered to the patient. A source of oxygen, a source of air and means connected to the source for mixing oxygen and air, a means for mixing being controlled by a calculation means and having an output adapted to be connected to the patient. The calculation means controls the oxygen concentration that the means for mixing feeds to the patient to cause the blood in the patient to reach a predetermined hemoglobin saturation level which adapts to the patient's requirements. These and other objects of the invention are achieved by providing an adaptive controller for delivering fractional inspired oxygen to a patient. The controller comprises a pulse oximeter connected by an optical sensor to the patient for measuring the patient's blood hemoglobin saturation and pulse rate. The pulse oximeter generates signals representative of the blood hemoglobin saturation and the pulse rate. Calculation means are provided which are responsive to the signals from the pulse oximeter for determining the fractional inspired oxygen level to be delivered to the patient. A source of oxygen and a source of air are provided for combining or mixing the oxygen and the air. The means for mixing is controlled by calculation means to provide a calculated percentage of oxygen and has an output connected to the patient so that the gas taken in by the patient automatically causes the blood in the patient to reach a predetermined hemoglobin saturation level which adapts to the patient's requirements.

Another object of the invention is to provide methods for therapeutic or diagnostic applications using a solenoid mixing system with an adaptive oxygen controller, using a pulse oximeter as a feedback signal for a patient in need of supplemental oxygen therapy. The patient in need of supplemental oxygen therapy maybe a neonate, a toddler, a school age child, a pre-teenager, a teenager or an adult. One therapeutic application for patient in need of supplemental oxygen therapy is for a patient that suffers from sleep apnea. The invention provides diagnostic analysis and therapy of patients suffering sleep apnea by monitoring blood oxygen levels and providing adjusting the fraction of inhaled oxygen and recording such adjustments of a sleep apnea episode. Another therapeutic application is for a patient that requires long-term supplemental oxygen therapy. The invention provides diagnostic analysis of a patients' oxygen requirement prior and during long-term supplemental oxygen treatment. During long-term supplement oxygen treatment, the invention provides therapy to the patient by adaptive adjustment of the inhaled gas mixture. Another therapeutic application is to gradually wean a patient from long-term supplemental oxygen therapy. Another therapeutic application is the adaptive adjustment of an oxygen-helium mixture of the breathing gas. Another object of the invention is the use of a continuous positive airway pressure by using an adaptive means of controlling a breathing gas mixture.

Other objects and many of the attendant advantages of this invention will be readily appreciated as the same becomes better understood by reference to the following detailed description when considered in connection with the accompanying drawing.

### BRIEF DESCRIPTION OF THE DRAWINGS

FIG. 1 is a diagrammatic representation of the bi-modal solenoid mixing system with the mixing chamber

FIG. 2 is a diagrammatic representation of the variable solenoid mixing system

FIG. 3 is a schematic block diagram of a solenoid mixing system with an adaptive controller of patient fractional inspired oxygen.

### DETAILED DESCRIPTION

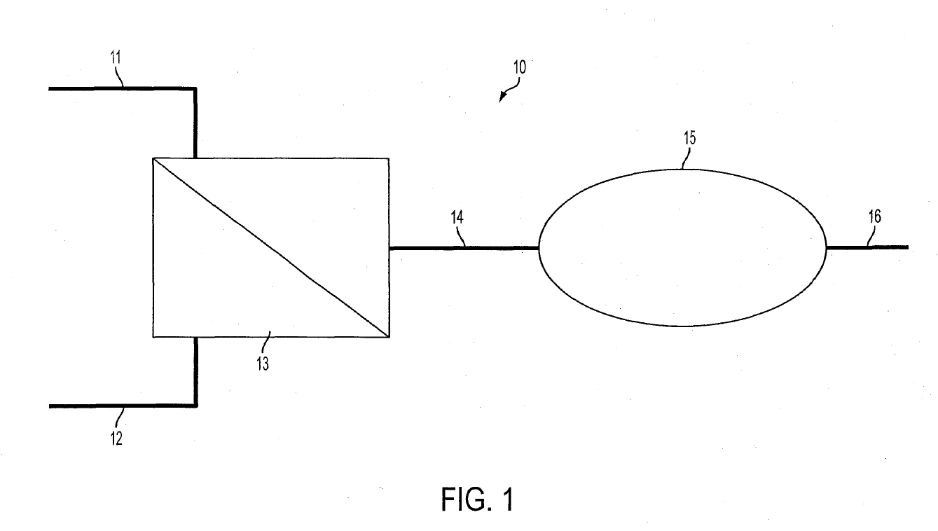

Referring now in greater detail to the figures and drawings, the input gases 11, 12 shown in FIG. 1 referred to generally as 10, are 21% oxygen, 11, and 100% oxygen, 12. The input gases are fed into the input ports of the bi-modal solenoid, 13. The output gas, 14, exits the bi-modal solenoid. A computer (not shown) that toggles between the two input gases determines the concentration of the output gas. The solenoid output gas, 14, is input into a mixing chamber, 15, where the output gas is mixed so to eliminate the pulsatile nature of the out gas from the bi-modal solenoid. After proceeding through the mixing chamber, the output gas, 16, exits the mixing chamber.

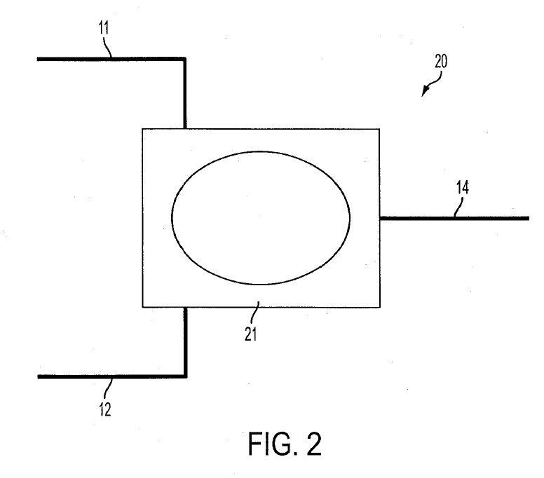

Referring to FIG. 2, referred to generally as 20 the input gases 11, 12, are 21% oxygen, 11, and 100% oxygen, 12. The input gases are fed into the input ports of the proportional solenoid, 21. The output gas, 14, exits the variable solenoid. A computer that varies between the 2 input gases determines the concentration of the output gas.

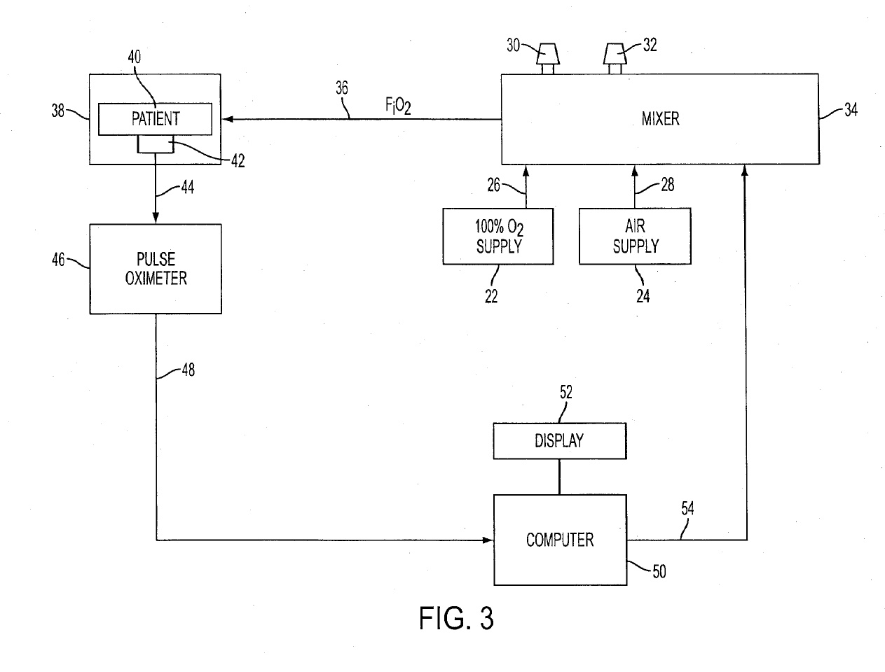

Referring now to FIG. 3, a solenoid system with an adaptive controller of patient fractional inspired oxygen for the purpose of providing fractional inspired oxygen to a patient 40, is shown in schematic block diagram.

A 21% oxygen source 22 is input via hose 26 to an oxygen/air mixer 34. A 100% oxygen source 24 is input via hose 28 to same oxygen/air mixer 34. Pressure regulators 30, 32 are used to control 21% oxygen and 100% oxygen input lines respectively. The mixer combines both 21% and 100% oxygen input gases by means of either bi-modal or variable solenoid system to form a fraction of inspired oxygen concentration (FiO₂) to the patients breathing tube 36.

A breathing tube 36 directs the gas mixture to the patient 40 via a nasal cannula or breathing mask 38.

An optical sensor 42 is placed on the patient's finger. The sensor, which may include a wrist strap for securing the sensor to the patient, extends from the patient 40 to a pulse oximeter 46 via a cable 44.

The system also includes a pulse oximeter 46 of the type made by Nellcor Incorporated, of Haywood, Calif. and which is described in U.S. Pat. No. 4,653,498 issued on Mar. 31, 1987. Pulse oximeter 46 is connected by a fiber optic cable 44 to the sensor 42.

The pulse oximeter is connected via a RS 232 cable 48 to a single board computer (SBC) 50. The SBC's output is connected by a RS 232 cable 54 to a mixer 34.

Finally, the patient's output data, control parameters, and alarm features are displayed on a flat screen module 52.

The present invention provides methods for therapeutic applications using a solenoid mixing system with an adaptive oxygen controller, using a pulse oximeter as a feedback signal for a patient in need of supplemental oxygen therapy. Such applications for a patient in need of supplemental oxygen therapy maybe a neonate, a toddler, a school age child, a pre-teenager, a teenager or an adult.

Therapeutic applications for patient in need of supplemental oxygen therapy is include patients that suffer from sleep apnea and those in need of long-term supplemental oxygen therapy who suffer from breathing disorders as well as gradually weaning those patients on long-term supplemental oxygen therapy away from the long-term supplemental oxygen therapy. Similarly, the supplemental oxygen therapy patient may require an oxygen helium mixture. The pulse oximeter feedback signal may also provide a continuous positive airway pressure.

Other objects and many of the attendant advantages of this invention will be readily appreciated as the same becomes better understood by reference to the following detailed description when considered in connection with the accompanying drawing Without further elaboration the foregoing claims will so fully illustrate my invention that others may, by applying current or future knowledge, adopt the same for use under various conditions of service.

I claim:

1. A solenoid mixing system that uses two input gases of 21% and 100% oxygen to produce an output gas that varies between 21% and 100% oxygen concentration.

2. The solenoid mixing system of according to claim 1, wherein the solenoid is a bi-modal solenoid.

3. The solenoid mixing system according to claim 1 wherein the solenoid is a proportional solenoid.

4. The bi-modal solenoid mixing system according to claim 2, wherein a computer toggles the bi-modal solenoid between two input gases to determine an output gas concentration.

5. The computer according to claim 4, wherein the computer uses a SpO₂ feedback to determine the precise oxygen supplemental concentration delivered to a patient.

6. The bi-modal solenoid mixing system according to claim 2, wherein a mixing chamber located between the bi-modal solenoid and the patent ensures complete mixing of an output gas.

7. The mixing chamber according to claim 6, wherein the mixing chamber eliminates a pulsatile nature of the output gas mixture from the bi-modal solenoid.

8. The variable solenoid mixing system according to claim 3, wherein the variable solenoid uses two precisely tuned input pressures to produce an output gas.

9. The proportional solenoid mixing system according to claim 1, wherein the input gases comprise 21% and 100% oxygen and the output gas varies between 21% and 100% oxygen concentration.

10. The proportional solenoid mixing system according to claim 3, wherein a computer varies the proportional solenoid between the two input gases to determine an output gas concentration.

11. A solenoid mixing system comprising a solenoid and an adaptive controller, wherein said adaptive controller is further employed for delivering fractional inspired oxygen to a patient, said adaptive controller comprising a pulse oximeter adapted to be connected by an optical sensor to said patient for measuring said patient's blood hemoglobin saturation and pulse rate, said pulse oximeter generating signals representative of said blood hemoglobin saturation and said pulse rate, calculation means responsive to said signals from said pulse oximeter for determining the fractional inspired oxygen level to be delivered to the patient, a source of oxygen, a source of air and means connected to said source for mixing oxygen and air, said means for mixing being controlled by said calculation means and having an output adapted to be connected to the patient, said calculation means controlling the oxygen concentration that said means for mixing feeds to the patient to cause the blood in the patient to reach a predetermined hemoglobin saturation level which adapts to the patient's requirements.

12. The adaptive controller according to claim 11, wherein said calculation means determines blood partial pressure from the signals provided by said pulse oximeter for enabling continuous adjustment of said patient's delivered fractional inspired oxygen percentage.

13. The adaptive controller according to claim 11, wherein said controller comprising a detection device adapted to be connected to said patient for measuring said patient's blood hemoglobin saturation and pulse rate, said device generating signals representative of said blood hemoglobin saturation and said pulse rate, calculation means responsive to said signals from said device for determining the fractional inspired oxygen level to be delivered to the patient, a source of oxygen and a source of air and means connected to said source for mixing oxygen and air, said means for mixing being controlled by said calculation means and having an output adapted to be connected to said patient, said calculating means controlling the oxygen concentration that said means for mixing feeds the patient to cause the blood in the patient to reach a predetermined hemoglobin saturation level.

14. A method of using a solenoid mixing system with an adaptive oxygen controller, comprising using a pulse oximeter as a feedback signal for a patient in need of supplemental oxygen therapy.

15. The method of using a solenoid mixing system with an adaptive oxygen controller according to claim 14, wherein the patient is a neonate, a toddler, a school age child, a pre-teenager, a teenager or an adult.

16. The method of using a solenoid mixing system with an adaptive oxygen controller according to claim 14, wherein the patient in need of supplemental oxygen therapy suffers from sleep apnea.

17. The method of using a solenoid mixing system with an adaptive oxygen controller according to claim 14, wherein the patient in need of supplemental oxygen therapy requires long-term supplemental oxygen therapy.

18. The method of using a solenoid mixing system with an adaptive oxygen controller according to claim 17, wherein the patient in need of requires long-term supplemental oxygen therapy is gradually weaned from the long-term supplemental oxygen therapy.

19. The method of using a solenoid mixing system with an adaptive oxygen controller according to claim 14, wherein the supplemental oxygen therapy comprises an oxygen helium mixture.

20. The method of using a solenoid mixing system with an adaptive oxygen controller according to claim 14, wherein the pulse oximeter feedback signal provides continuous positive airway pressure.
